(19) United States
(12) Patent Application Publication
(10) Pub. No.: US 2025/0049733 A1
(43) Pub. Date: Feb. 13, 2025

(54) PHARMACEUTICAL COMPOSITION FOR THE TREATMENT OF INFECTIOUS RESPIRATORY DISEASES CAUSED BY INFLUENZA AND SARS-COV-2, AMONG OTHERS

(71) Applicant: ALPARIS, S.A. DE C.V., Mexico City (MX)

(72) Inventors: Gustavo BARRANCO HERNÁNDEZ, Mexico City (MX); Héctor SENOSIAIN ARROYO, Mexico City (MX)

(73) Assignee: ALPARIS, S.A. DE C.V., Mexico City (MX)

(21) Appl. No.: 18/721,048

(22) PCT Filed: Dec. 16, 2022
(86) PCT No.: PCT/IB2022/062339

§ 371 Date: Jun. 17, 2024

(30) Foreign Application Priority Data
Dec. 17, 2021 (MX) MX/A/2021/016108

(51) Int. Cl. A61K 31/12; A61K 9/00; A61K 31/14; A61K 31/198; A61K 36/9066; A61K 47/10; A61K 47/32; A61K 47/36; A61K 47/38; A61P 31/14

(52) U.S. Cl. CPC A61K 31/12; A61K 9/0078; A61K 31/14; A61K 31/198; A61K 36/9066; A61K 47/10; A61K 47/32; A61K 47/36; A61K 47/38; A61P 31/14

(57) ABSTRACT

The present invention relates to a pharmaceutical composition for nasal delivery comprising a co-amorphous compound of curcumin and arginine for use in the treatment of infectious respiratory diseases and airborne diseases such as the respiratory infection caused by SARS-CoV-2, influenza virus, respiratory syncytial virus and adenovirus, among others.

### FIELD OF INVENTION

The present invention relates to the field of pharmaceutical compositions based on pharmaceutical compounds, the treatment of infectious diseases, infectious respiratory diseases and airborne diseases, as well as nasal delivery formulations. More specifically, the present invention refers to the combination of arginine and curcumin, its topical and nasal delivery, its use for the treatment of infections, as well as a pharmaceutical composition in solution, gel or microemulsion.

### BACKGROUND OF THE INVENTION

Increasing microbial resistance to antibiotics and antivirals jeopardizes the effectiveness of the prevention and treatment of infections by viruses, bacteria, fungi and parasites. Infectious respiratory diseases and airborne infections are a public health problem. Viruses such as influenza, coronaviruses that cause SARS and MERS have caused epidemics and pandemics at a high cost, thus their control, prevention and treatment constitute worldwide health challenges. Although there are many alternatives to treat different types of infectious respiratory diseases and airborne infections, additional therapies are still required to help solve this problem.

The nasal administration of therapeutic agents represents an alternative that is not only viable, but also advantageous in the context of particular therapies, such as those focused on diseases of the respiratory system, including infectious diseases. The nasal cavity is an easily accessible organ, covered with mucous membranes that are highly vascularized and contains numerous microvilli, providing a large surface area available for the rapid absorption of therapeutic molecules, eliminating hepatic metabolism and the hostile gastrointestinal environment. Therefore, the nasal route of administration is ideal for drugs whose site of action is the nasal mucosa and which do not achieve the therapeutic concentrations when administered systemically. Such is the case of curcumin, because its low solubility, permeability and rapid metabolism limit its therapeutic action. Additionally, an easy access to the nasal route can generate better patient adherence to treatment, which is critical to obtain the expected result.

However, there are several challenges in the development of nasal formulations. High molecular weight compounds are not easily administered by the nasal route; problems of dispersion and absorption on the nasal surfaces occur frequently; residence times of the active ingredients may be short; there may be a lack of precision in the dose administered; the volumes to be administered are limited; the components of the administered compositions may cause local irritation and other potential side effects.

Curcumin is the main active metabolite of the plant Curcuma longa, commonly known as turmeric. The systematic name for curcumin is (1E,6E)-1,7-bis(4-hydroxy-3-methoxyphenyl)-1,6-heptadiene-3,5-dione. Several studies about the biological and pharmacological activity of curcumin confirm its antitumor, anticancer, antioxidant, anti-arthritis, neuroprotective, anti-inflammatory, antifungal, antibacterial and antiviral properties of this compound, so it can be used for the treatment of infections caused by different etiologic agents. Specifically, curcumin has been studied for its antiviral action against SARS-CoV2 viruses, Respiratory Syncytal Virus (RSV), influenza, Hepatitis and Herpes simplex virus, among others, for which a respective IC50 of 7.9 mcg/mL, 10 mcg/mL, 0.17 mcg/mL, 1.68 mcg/mL, 11.05 mcg/mL was found. However, despite the therapeutic potential of curcumin, its medical use has been found difficult due to its low solubility in water, as well as its low bioavailability; only 1% of curcumin is absorbed systemically by the body, and after a half-life of about 8 hours, it is degraded into several ineffective products. This was corroborated in a pilot pharmacokinetic study with a crossover design with 2 periods and 2 sequences in 18 healthy volunteers, using two capsules of a food supplement widely known in Mexico, which in total contains 1000 mg of phytosomal turmeric, equivalent to 150 mg of curcumin. The Cmax of free curcumin, glucuronide curcumin, and curcumin sulfate were 0.043±0.040, 16.723±9.381, and 0.989±0.798 ng/ml, respectively. These concentrations are too low to have an antiviral effect if curcumin is administered systemically, since they are below the mentioned IC50 values by several orders of magnitude. The same is corroborated by FIG. 1 of this application, which shows that plasma concentrations of free curcumin, provided by a commercial orally administered product, are in the order of ng/ml.

In order to solve the problem of solubility and bioavailability of curcumin, different alternatives have been proposed. For example, in document US20130273140A1, curcumin is encapsulated in a tetradecylmaltoside hydrogel; CN104997727B provides solutions comprising curcumin micelles where a peptide is used to modify the micelle; US20110034564A1 describes water-soluble complexes of curcumin and cyclodextrin; co-administration of an agent that improves the solubility of curcumin has also been proposed. Finally, U.S. Pat. No. 9,012,490B2 discloses curcumin analogues that are lipophilic so they can be targeted to the brain. Consequently, the formulation of curcumin-containing compounds in therapeutically effective amounts and with desirable characteristics remains a challenge. In addition, although state-of-the-art oral formulations of curcumin have been developed, to the best of the inventors' knowledge, none of them provide the concentrations necessary to inhibit etiologic agents causing infectious diseases, in particular, infectious respiratory diseases such as SARS.

On the other hand, different solid forms of an active ingredient may have improved chemical, biological, or physical properties, such as improved solubility, dissolution rate, bioavailability, pharmacokinetics, mechanical strength, flow properties, particle size, melting point, among others. In the case of curcumin, although WO2021044231A describes solid forms of curcumin and arginine with anti-inflammatory properties, it does not contemplate nasal administration, it does not describe specific compositions for the treatment of infectious diseases, infectious respiratory diseases and airborne infections, nor does it foresee the problems inherent in the development of topical medicaments and/or specific medicaments to treat these types of infections.

On the other hand, nitric oxide is known to have a broad antimicrobial effect against bacteria, fungi, helminths, protozoa and viruses, mainly due to damage to genetic material. However, its administration as a therapeutic agent is difficult because it is highly lipophilic and reacts quickly with molecular oxygen to generate nitrogen dioxide, which is toxic in low concentrations.

Patent documents US20210252043, U.S. Pat. Nos. 9,730,956 and 10,905,712 describe solutions that release gaseous nitric oxide to treat infections or wounds, including its administration into the respiratory tract. However, the solutions in such documents are based on the release of nitric oxide from nitrites at acidic pH, so these types of compositions are not suitable for nasal administration. In addition, they do not contemplate the use of arginine as an antimicrobial agent or the possible effects that could be achieved by combining it with curcumin.

Although compositions that may include curcumin and L-arginine have been described, neither of them contemplates both components as active ingredients. US20200352856A1 describes a composition for topical application, where curcumin is used as an anti-inflammatory, while L-arginine is used as a neutralizing agent; CN104997727B describes a solution comprising curcumin micelles, in which arginine is part of a peptide whose function is to modify the micelle. These documents also fail to address the challenges of the nasal formulation of curcumin and arginine, nor the benefits of using such a combination in the treatment of infectious diseases, infectious respiratory diseases and airborne infections.

Despite known state-of-the-art efforts, there remains a need to provide new therapeutic alternatives for the treatment of infectious diseases, infectious respiratory diseases and airborne infections, since there is a multiplicity of pathophysiological responses induced by etiological agents.

For this reason, the present invention proposes to administer curcumin in a nasal solution and in combination with arginine, that enhances its solubility, permeability and antiviral action by acting as a precursor of nitric oxide.

### BRIEF DESCRIPTION OF THE FIGURES

FIG. 1. Graph showing the plasma concentration (ng/ml) of free curcumin achieved with an oral dose of 150 mg curcumin from a commercial orally administered product.

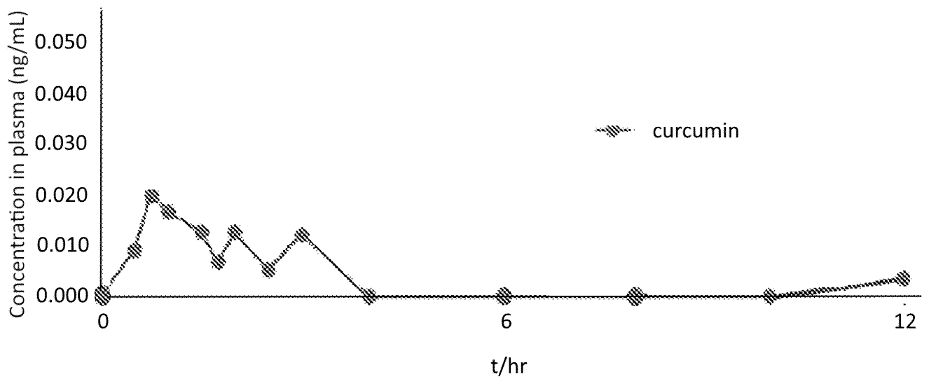

### DETAILED DESCRIPTION OF THE INVENTION

The present invention is based on the discovery that the combination of curcumin and arginine is advantageously effective in the treatment of infections, and especially in the treatment of infectious respiratory diseases and airborne infections. In addition, the present invention is based on the discovery that the nasal administration of curcumin and arginine is particularly useful in the context of respiratory tract infections and airborne infections.

Therefore, the present invention concerns the use of the combination of curcumin and arginine for the treatment of infections and specifically, infectious respiratory diseases and airborne infections. The present invention also concerns a nasal pharmaceutical composition comprising curcumin and arginine, useful for the treatment of infectious respiratory diseases.

For purposes of this disclosure, the term “combination” encompasses the administration of curcumin and arginine to a patient to treat a disease, by the same or different routes of administration and by the same or different dosage forms. It also includes administering curcumin and arginine at the same time or at different times.

Curcumin is the main active metabolite of the rhizome of the plant Curcuma longa, commonly known as turmeric. Curcumin is an orange-yellow solid with a melting point of 183° C., soluble in ethanol and concentrated acetic acid. It decomposes at high temperatures and is photosensitive. As for its chemical nature, it is a diarylheptanoid compound belonging to the curcuminoid group. The IUPAC name for curcumin is (1E,6E)-1,7-bis(4-hydroxy-3-methoxyphenyl)-1,6-heptadiene-3,5-dione. There are two tautomeric forms of curcumin, which are the keto form and the enol form. Formulas I and II represent the keto and enol forms of curcumin, respectively.

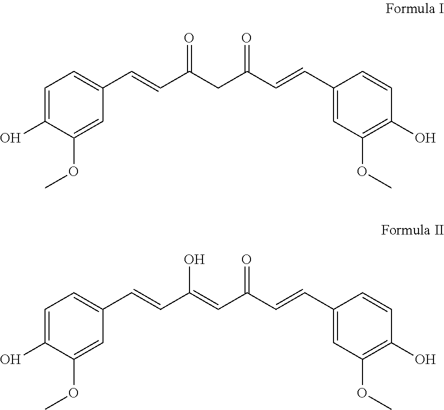 [SMILES: COc1cc(/C=C/C(=O)CC(=O)/C=C/c2ccc(O)c(OC)c2)ccc1O ; COc1cc(/C=C/C(=O)/C=C(O)/C=C/c2ccc(O)c(OC)c2)ccc1O]

Regarding its use in the treatment and prophylaxis of infections, it has been found that curcumin has different mechanisms of action against bacteria, such as inhibition of DNA replication, damage to the cell membrane, motility reduction, stimulation of an apoptosis-like response, and modification of GTPase activity in the cytoskeleton, so that it has activity against Gram-positive and Gram-negative bacteria, such as, but not limited to, Staphylococcus aureus, Streptococcus pyogenes, enterotoxigenic Escherichia coli, Pseudomonas aeruginosa, among others. It has also been described that it can intervene in viral entry, viral replication, viral protein expression, viral assembly, and virion release. Due to the variety of mechanisms, curcumin and its derivatives have shown antiviral activity against a large number of viruses, including, but not limited to, dengue virus, Zika virus, chikungunya virus, human immunodeficiency virus (HIV), hepatitis virus, human norovirus, influenza virus, herpes bovine virus, respiratory syncytial virus, Japanese encephalitis virus, Epstein-Barr virus, human cytomegalovirus, coronaviruses that cause severe acute respiratory syndrome, including SARS-CoV-2.

Randomized controlled trials have shown that curcumin significantly reduces the secretion of tumor necrosis factor-alpha (TNF-α) and interleukin-6 (IL-6). Likewise, a meta-analysis showed that 8 weeks of treatment with 1 g of curcumin daily can reduce symptoms associated with inflammation in rheumatoid arthritis.

On the other hand, arginine (Formula III) is one of the twenty amino acids that are part of proteins and is classified as a semi-essential amino acid. L-arginine is preferably used in the present invention.

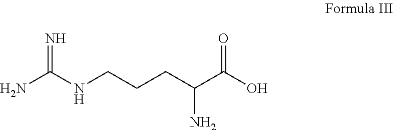 [SMILES: N=C(N)NCCCC(N)C(=O)O]

Arginine is involved in the mechanism of vascular synthesis of nitric oxide; nitric oxide production in epithelial and endothelial cells is closely linked to L-arginine uptake from the extracellular medium. Nitric oxide synthase (NOS) enzyme catalyzes the conversion of L-arginine to L-citrulline by oxidation dependent from NADPH and tetrahydrobiopterin (BH4), generating nitric oxide as one of the reaction products. In humans there are three NOS isoforms: neuronal (NOS, NOS1), endothelial (eNOS, NOS3) and inducible (iNOS, NOS2), which are found in the paranasal mucosa, with the paranasal sinuses being the main source of intrinsic nitric oxide production in the respiratory tract. In addition, the production of nitric oxide from L-arginine by the nitric oxide synthase enzyme has been shown to be essential for the innate immune response, so the availability of arginine plays an important role in host resistance to infection.

Nitric oxide has been linked to body functions such as vasodilation, regulation of immune responses against infections, and has been recognized due to its antimicrobial ability to fight bacterial, viral, and fungal agents. Depending on its concentration, nitric oxide exerts antimicrobial activity in two ways. At low concentrations it acts as a signaling molecule promoting the growth and activity of immune system cells, while at high concentrations it covalently binds to DNA, proteins and lipids, eliminating pathogens. Whilst not being bound by theory, nitric oxide exerts antiviral effects on DNA and RNA viruses such as adenoviruses, coronaviruses, influenza viruses, parainfluenza viruses, among others. In addition, nitric oxide treatments are known to improve oxygenation and reduce pulmonary infiltrates associated with viral infections. Due to the various mechanisms of action, nitric oxide is effective against a wide variety of bacteria, including multidrug-resistant bacteria such as Staphylococcus aureus, Escherichia coli, Pseudomonas aeruginosa, bacterial and fungal biofilms, and even bacterial spores.

The inventors unexpectedly discovered that the combination of the present invention has advantageous and unexpected effects, as it provides anti-inflammatory, antibacterial, antiviral, antifungal, antithrombotic, anticoagulant effects, as well as improvements in the innate immune response. Specifically, the combination of curcumin and arginine is particularly advantageous against viral infections, such as dengue virus, Zika virus, chikungunya virus, human immunodeficiency virus (HIV), hepatitis virus, human norovirus, influenza virus, respiratory syncytial virus, Japanese encephalitis virus, Epstein-Barr virus, herpes virus, Coxsackie virus, Hantavirus, human cytomegalovirus, and coronaviruses that cause severe acute respiratory syndrome (SARS) and Middle East Respiratory Syndrome (MERS), rhinovirus, adenovirus, enterovirus; bacterial infections caused by a variety of bacteria including but not limited to Streptococcus pneumoniae, Mycoplasma pneumoniae, Streptococcus pyogenes, Staphylococcus aureus, Streptococcus pneumoniae, Haemophilus influenzae, Escherichia coli, Pseudomonas aeruginosa, among others.

Therefore, the present invention is an effective alternative to the treatment of infectious diseases caused by bacteria and viruses. Without claiming to limit the scope of the invention by theory, it is believed that the combination of the invention may act by inhibiting stages of viral replication cycles and the bacterial cell cycle, improving the innate immune response in early stages of infections and in severe cases, decreasing complications of infectious diseases.

In some respects, the combination of the present invention is useful in the treatment of infections on mucocutaneous surfaces, including infections caused by agents entering the body through such surfaces. Mucocutaneous surfaces may include the oral cavity, nasal cavity, eyes, rectal tissue, vaginal tissue, bladder, urinary tract, and wounds (such as skin wounds).

In preferred aspects, the combination of the present invention is useful in the treatment of infectious respiratory diseases and airborne infections, including disease symptomatology and complications.

As defined herein, an infectious respiratory disease is an infection caused primarily by viruses, bacteria, or fungi. The infectious respiratory diseases subject to the present invention include the common cold, rhinitis, pharyngitis, tonsillitis, epiglottitis, laryngotracheitis, influenza, Middle East Respiratory Syndrome, Acute Respiratory Syndrome, in particular COVID-19. The symptoms that may be treated with the present invention include those related to inflammatory processes such as swelling, redness and pain.

The combination of the invention is useful for the treatment and prophylaxis of infectious diseases. In additional respects, infectious diseases are preferably infectious respiratory diseases and airborne infections.

In one embodiment, infectious respiratory disease is selected from the group consisting of the common cold, rhinitis, pharyngitis, tonsillitis, epiglottitis, laryngotracheitis, influenza, Middle East Respiratory Syndrome, Acute Respiratory Syndrome, particularly COVID-19. In one embodiment, the etiologic agent is a virus that is selected from the group consisting of rhinovirus, coronavirus, parainfluenza virus, Epstein-Barr virus, cytomegalovirus, herpes simplex virus, adenovirus, influenza virus, respiratory syncytial virus, MERS coronaviruses, enteroviruses, SARS-CoV-1, and SARS-CoV-2. In one embodiment, the etiologic agent is a bacterium selected from the group consisting of Mycoplasma pneumoniae, group A streptococci, Mycoplasma hominis, Streptococcus pneumoniae, Corynebacterium diphtheriae, Streptococcus pyogenes, and Haemophilus influenzae.

In another embodiment, the combination of the present invention relieves, reduces or suppresses symptoms of infections associated with inflammatory processes. In one embodiment, the symptoms are associated with wound infections, selected from swelling and redness of the affected area, pain and burning. In a preferential embodiment, symptoms are associated with infectious respiratory diseases, selected as headache, pain and/or burning of the throat, head and ear.

In an additional embodiment, the combination of the invention decreases the risk of complications associated with selected bacterial or viral infections of hyperinflammatory syndrome and procoagulant and prothrombotic events. In a preferred embodiment, the combination of the invention can be used for the treatment of hyperinflammatory syndrome in patients with severe infectious respiratory disease. In a still more preferred embodiment, the combination of the invention reduces the secretion of pro-inflammatory cytokines. In another embodiment, the combination of the invention decreases the risk of procoagulant and prothrombotic events in hospitalized patients due to inhibition of platelet aggregation and thromboxane A2 (TXA2) formation by platelets.

In one aspect, the combination of the invention can be used in the treatment and prevention of COVID-19 disease caused by SARS-CoV-2 due to the following mechanisms:

Inhibition of viral entry into cells by interaction of curcumin with the S1 glycoprotein of SARS COV-2.

Inhibition of viral entry into cells by binding curcumin to angiotensin-converting enzyme-2 (ACE-2) widely expressed in cells of the lungs, heart, liver, vascular endothelium, kidneys, and intestine.

Inhibition of viral entry into cells by interaction of curcumin with transmembrane protease serine 2 (TMPRSS2) that facilitates SARS-CoV-2 entry into cells.

Decreased viral load due to inhibition of the main SARS-CoV-2 protease (Mpro) provoked by its interaction with curcumin.

Decreased viral load due to inhibition of RNA-protease dependent from RNA polymerase (RdRp) of SARS-CoV-2, caused by its interaction with curcumin.

Reduction of viral replication by nitrosation (nitrosonium NO+ donation) of viral proteins and cellular factors required in viral replication.

Reduction of virion formation efficiency by interaction of curcumin with one of the helices of the SARS-CoV-2 E protein that is part of the viral envelope.

Reduction of viral infectivity by increasing the mutation rate by the interaction of free nitric oxide in solution and the superoxide anion.

In another aspect, the combination of the invention can be used in the treatment and prevention of influenza due to the following mechanisms:

Inhibition of viral entry into cells by interaction of curcumin with hemagglutinin (HA).

Reduction of viral replication by inhibition of neuraminidase (NA) activity.

Reduction of the efficiency in virion formation by inhibiting viral nucleoprotein nucleation.

Reduction of viral infectivity due to its binding to hemagglutinin (HA) and by inhibiting the expression of the MDM2 protein.

In an additional aspect, the combination of the invention can be employed in the treatment and prevention of respiratory syncytial virus due to the following mechanisms:

Reduction of viral replication by increasing phosphorylation of the α subunit of eukaryotic translation initiation factor 2 (eIF-2α) and protein kinase R (RPKR) expression in cells, as well as by inhibiting proteasomes.

Reduction of virion formation efficiency by inhibition of viral nucleoprotein nucleation.

Increased innate immune response, increased epithelial barrier of the upper respiratory tract.

For purposes of this disclosure, the terms “treating” and “treatment” include alleviating, lessening, ameliorating and suppressing at least one symptom of a disease or condition, preventing the occurrence of additional symptoms, preventing the aggravation of the disease (i.e., preventing the aggravation of the disease during the course of the same), and inhibiting the disease or condition. Therefore, it is intended that these terms include both the therapeutic treatment of the disease or condition, including early and late phases of the disease, and prophylactic treatment. For example, prophylactic treatment may refer to treatment designed to be given before the onset of symptoms of an infection, when contact with infected people is suspected, or for decreasing the risk of acquiring a wound infection, or an airborne infection. In some aspects, prophylactic treatment can increase the effectiveness of a patient's immune response to viruses and/or bacteria.

In certain aspects, the combination of the present invention includes curcumin and arginine as active ingredients. In this way, the combination of the invention can provide a therapeutic effect without the need to incorporate an additional active agent, such as an antiviral agent, an antibacterial agent, an anti-inflammatory agent, an antihistamine, or some other agent useful for the treatment of a respiratory tract disease. However, the combination of the present invention may also include some additional active agent.

Curcumin and arginine, as components of the inventive combination, can be incorporated as any chemical form of the respective substances. For example, the invention contemplates the use of curcumin and arginine in free form, pharmaceutically acceptable salts thereof, curcumin and arginine complexes, crystalline or amorphous solid compounds comprising curcumin and arginine, and mixtures thereof. According to the present invention, a pharmaceutically acceptable salt is one that, according to reasonable medical judgment, is acceptable for administration to the human or animal body without an undesirable complication or response, with a reasonable risk-benefit ratio. Pharmaceutically acceptable salts within the scope of the present invention possess the desired therapeutic activity, and include, for example, addition salts with organic acids, with organic bases and inorganic bases.

In preferred respects, the combination of the present invention comprises a solution or a microemulsion formed from a co-amorphous solid compound of curcumin and arginine (preferably L-arginine). The molar ratio of curcumin:arginine in co-amorphous can range from 1:5 to 5:1, preferably from 1:2 to 2:1, and most preferentially 1:2.

In a favorite aspect of the invention, the co-amorphous compound is composed of curcumin and arginine in a molar ratio of 1:2. Such co-amorphous compound can be prepared by dissolving a 1:2 molar mixture of curcumin and arginine in a solvent, such as ethanol; then slowly distilling the solvent until a minimum volume of solvent is obtained, and finally carrying out rapid evaporation of the remaining solvent (distillation/evaporation can be done in rotary evaporator by decreasing the pressure), thus obtaining a solid that is the co-amorphous compound of curcumin and arginine.

The combination of curcumin and arginine of the present invention can be provided by means of a pharmaceutical composition. Such a pharmaceutical composition preferably comprises a pharmaceutically acceptable excipient and may be formulated for administration by any suitable route, including sublingual, buccal, topical, transdermal and nasal route. In other preferred respects, the combination is suitable for topical administration, including mucocutaneous surfaces that may include the oral cavity, nasal cavity, eyes, rectal tissue, vaginal tissue, bladder, and urinary tract, as well as wounds, such as skin wounds. More preferably, the combination is provided by means of a nasal pharmaceutical composition.

Indeed, the inventors surprisingly discovered that nasal administration of curcumin and arginine is advantageously useful, achieving an effective supply of these active ingredients to obtain the desired therapeutic effect. The nasal supply of curcumin and arginine is especially useful for treating infectious respiratory diseases and airborne infections.

Accordingly, the present invention also relates to a nasal pharmaceutical composition comprising the combination of curcumin and arginine as described. Such a composition is particularly useful for the treatment of infections, infectious respiratory diseases and airborne infections; however, its therapeutic application can be extended to any disease or condition that could benefit from the therapeutic effects of the invention's combination. Preferably, the nasal pharmaceutical composition is a nasal solution.

According to the present invention, nasal administration refers to the application of the pharmaceutical combination or composition of the invention to the upper respiratory tract by means of the nostrils. The upper airway includes the nasal cavity, pharynx, and larynx.

As is known by a technician in the field, mucosae, or mucous membranes, are epithelial linings covered by mucus. Mucous membranes are often affected by bacterial or viral infections. In particular, nasal mucosa contains a high density of the transmembrane protein ACE2 (Angiotensin Converting Enzyme II), which is employed by the SARS-CoV2 virus to enter host cells. Therefore, the composition of the present invention provides better means of administering therapeutically effective active agents to this membrane. In some respects, the pharmaceutical composition is applied to the vaginal, rectal, and nasal mucosae. In certain respects, the pharmaceutical composition is preferably applied to the nasal mucosa. The nasal mucosa is supplied by several blood vessels, ensuring rapid absorption of the administered drugs, formulations or compositions. In addition, an important aspect of nasal administration is that it is considered a non-invasive route of administration for easy application or self-application.

The concentration of curcumin in the composition may vary from 0.0001% to 1% by weight of the composition, such as from 0.001% to 0.5%, from 0.001% to 0.03% and from 0.001% to 0.01%. Preferably, the amount of curcumin varies between 0.001% and 0.05%, and specifically from 0.02% to 0.1%, including 0.03% to 0.1%. The weight percentages mentioned are based on the total weight of the composition.

The concentration of arginine in the composition may vary from 0.0001% to 1% by weight of the composition, such as from 0.001% to 0.5%, from 0.001% to 0.03% and from 0.001% to 0.01%. Preferably, the amount of curcumin varies between 0.001% and 0.05%, and specifically from 0.002% to 0.02%, including 0.03% to 0.1%. The weight percentages mentioned are based on the total weight of the composition.

The use of curcumin and arginine co-amorphous compound provides additional advantages that are particularly useful in the context of nasal administration, such as improved solubility and permeability with respect to curcumin, better bioavailability than pure curcumin or phytosomal curcumin, and an anti-inflammatory action as potent as some NSAIDs in an acute and subchronic inflammation model.

When the pharmaceutical composition of the present invention uses a solid form of curcumin and arginine, such as a co-amorphous compound in a molar ratio of 2:1, such solid form may be found in a concentration between 0.0001% and 1%, such as between 0.0005% and 0.05%, preferably between 0.001% and 0.02%, more preferably between 0.03% and 0.02%. Particularly, the amount of the solid form is 0.001%, 0.002%, 0.003%, 0.004%, 0.005%, 0.006%, 0.007%, 0.008%, 0.009%, 0.01%, 0.02%, 0.03%, 0.04 or 0.05%. The weight percentages mentioned are expressed based on the total weight of the composition.

Importantly, if the pharmaceutical composition of the invention is formulated as a solution, the curcumin-arginine co-amorphous compound may partially dissociate in the solution. In such a case, however, the advantageous effects of the invention described here are preserved.

The composition of the present invention may contain one or more pharmaceutically acceptable excipients. Within the scope of this invention, a pharmaceutically acceptable carrier is a material, composition, or vehicle involved in carrying or transporting an active ingredient, and which, in reasonable medical judgment, is acceptable for administration to the human or animal body without an undesirable complication or response with a reasonable risk-benefit ratio. Examples of pharmaceutically acceptable excipients include vehicles, solubilizers, emulsifiers, binders, preservatives, mucoadhesives, surfactants, lubricants, viscosants, dyes, pH buffers, stabilizers, and/or adjuvants.

In certain respects, vehicles can be selected from one or more of isotonic saline solution, water and seawater. In additional aspects, vehicles are preferably selected from isotonic saline solution and water. In certain respects, water or isotonic saline solution is present in an amount of at least about 50%, about 55%, about 60%, about 65%, about 70%, about 75%, about 80%, about 85%, about 90%, about 95%, or about 99.99% by volume of the total volume of the composition. In some embodiments, water or saline solution is present in an amount of about 90%, about 91%, about 92%, about 93%, about 94%, about 95%, about 96%, about 97%, about 98%, about 99%, about 99.99, or about 99.999% by volume of the total volume of the composition.

In some additional respects, surfactants may be selected from one or more of propylene glycol, polyethylene glycol, glycerin, diethylene glycol monoethyl ether (trascutol), polyoxyethylene-polyoxypropylene (lutrol), cetylpyridinium chloride (CPC), cetylpyridinium bromide (CPB), Tween 80, Poloxamer 407, and combinations thereof.

In certain respects, the pharmaceutical composition comprises one or more surfactants that can be selected from polyethylene glycol, PEG400, PEG3500, polyoxyl stearate 400, polysorbate 20, polysorbate 80, propylene glycol, glycerin, lutrol and trascutol. In certain respects, the surfactant is preferably glycerin and/or propylene glycol. In some respects, the surfactant is present in sufficient quantity to give stability to the active agent. In certain aspects, the surfactant is present in a concentration of up to 1%, up to 2%, up to 3%, up to 4% up to 5%, up to 10% by weight of the composition.

In certain respects, mucoadhesives may be selected from one or more of methyl vinyl ether copolymer, hydroxypropyl methylcellulose (HPMC or hypromellose), sodium carboxymethylcellulose, carbopol, alginate salts (sodium, potassium, magnesium alginate), modified starches, polyvinyrrolidone, chitosan, and Eudragit RL-100. In some additional respects, mucoadhesives are preferably selected from one or more of HPMC, carbopol, sodium alginate, and combinations thereof.

In some respects, the pharmaceutical composition is a solution, a gel or a dispersion or a microemulsion. Preferably it is a solution. In additional aspects, the pharmaceutical composition is adapted to be administered by means of droppers, sprays, pressurized atomizers, inhalation devices, among others. Preferably, it can be administered by spray.

In preferred aspects, the pharmaceutical composition of the invention is a solution suitable for atomization in the nasal cavity. This dosage form has the advantages of achieving sufficient and homogeneous coverage of the surface of the nasal cavity, which allows the active ingredients to be delivered effectively.

When formulated as a solution, the pharmaceutical composition is preferably isotonic. Within the scope of the present invention, “isotonic” is a solution whose osmotic concentration varies between 280 and 315 mOsm/L. The isotonicity of the solution prevents undesirable reactions in the nasal mucosa.

Also, the solution preferably has physiological pH. For this purpose, the composition may include a buffering agent to maintain a pH in the solution of 6 to 8, preferably around 7.0, such as 6.5-7.5 and more preferentially from 6.5 to 7.0. Preferably, the pH of the solution should be such as to avoid undesirable reactions in the nasal mucosa. A buffering agent may be used to adjust the pH of the composition to the above ranges. In certain aspects, the buffering agent can be selected from one or more of phosphate salts, carbonate salts, and combinations thereof. Preferably, the buffering agent is a phosphate salt, such as KH₂PO₄, K₂HPO₄ and/or Na₂HPO₄.

However, it is well known that the pH of compositions or formulations can affect the stability of the active agent(s). This is of particular relevance to the present invention since, during its development, the inventors observed that the co-amorphous compound of arginine and curcumin alkalizes the pH of the composition exceeding the suggested range for nasal administration compositions, pH 5-6. In addition, the inventors noticed that, when adjusting the pH of the composition to levels of 5-7, the curcumin-arginine co-amorphous compound loses stability. The inventors have solved this problem by adding one or more surfactants to the composition.

In certain respects, the pharmaceutical composition comprises one or more mucoadhesive agents, useful for increasing the contact time of the pharmaceutical with the mucous membranes. In addition, one or more mucoadhesives are present in an effective amount to allow the composition to cover the mucocutaneous area. In certain respects, the one or more mucoadhesives are present in a concentration of approximately 0.001% to approximately 10%, wherein the concentration of one or more mucoadhesives is a function of the mucoadhesive(s) used. In some respects, the composition comprises a mucoadhesive. In additional respects, the composition comprises more than one mucoadhesive. In certain respects, the one or more mucoadhesives can be selected from methyl vinyl ether copolymer, hydroxypropyl methylcellulose (HPMC or hypromellose), sodium carboxymethylcellulose, carbopol, alginate salts, modified starches, polyvinyrrolidone, beta-glucans, chitosan, and Eudragit RL-100. In additional aspects, one or more mucoadhesives are preferably selected from HPMC, carbopol and sodium alginate.

In addition, the present invention provides the use of a combination comprising curcumin and arginine in the manufacture of a pharmaceutical composition for the treatment and prophylaxis of infectious diseases, preferably infectious respiratory diseases and airborne infections, symptoms and complications as described above. Such a pharmaceutical composition can be or can form part of a medicament. Accordingly, the present invention also refers to a method for treating an infectious respiratory disease and/or airborne infections, the method comprising administering a combination of curcumin and arginine, as described, to a patient suffering from such disease.

The nasal pharmaceutical composition can be administered at least once a day, wherein the administration includes one or two applications in each nostril. In particular, the pharmaceutical composition or medicament can be administered two, three, four, five, six or seven times a day. In particular, the pharmaceutical composition is administered to each nostril three to six times a day to treat the infectious diseases described above, wherein administration comprises one or two applications to each nostril. Preferably, nasal composition treats an infectious respiratory disease whose etiologic agent is a bacterium and/or a virus as described. In an even more preferred embodiment, the etiologic agent is SARS-CoV-2.

In another embodiment, the pharmaceutical composition is administered in each nostril one to three times a day for the prophylaxis of previously described infectious diseases, wherein the administration includes one or two applications in each nostril. In a preferred embodiment, infectious disease is an infectious respiratory disease whose etiologic agent is a bacterium and/or a virus as described. In an even more preferred embodiment, the etiologic agent is SARS-CoV-2.

The pharmaceutical composition is administered in an amount of 90 to 180 μL, preferably in an amount of 100 to 140 μL, into the nasal cavity.

Therefore, the present invention relates to a nasal pharmaceutical composition, as described, for use in the treatment and prophylaxis of infectious diseases, preferably infectious respiratory diseases and airborne infections, symptoms and complications as described above.

Concentrations, amounts and other numerical data are expressed or presented in a range format. It should be understood that such interval formatting is used simply for convenience and brevity and should therefore be interpreted flexibly to include not only the numeric values explicitly expressed as the interval boundaries, but also to include all the individual numeric values or secondary values included in the interval as if each numeric value and sub-interval were explicitly recited.

The examples described below are merely illustrative of the principles underlying this invention, as well as some embodiments thereof, and do not in any way limit the scope of the invention as described and claimed. These examples, together with the disclosure of the present invention and the figures, will make it clear to experts in the art how the various embodiments of the invention can be put into practice.

### Example 1. Composition of Nasal Spray Solution

Table 1 illustrates the components and concentrations that can be included in a nasal spray solution.

TABLE 1
Nasal Spray Composition

| Components | Contents in % of composition |
|---|---|
| Curcumin-L-Arginine co-amorphous | 0.01 to 0.10 |
| compound | |
| Surfactant 1 | 0.1 to 20 |
| Surfactant 2 | 0.1 to 20 |
| pH Regulator | 0.1 to 5 |
| Surfactant 3 | 0.1 to 20 |
| Vehicle | q.s. |
| Other additives | q.s. |
| Total Composition | 100% |

If required, an antibacterial agent, such as benzalkonium chloride, can be added in an amount of 0.1% of the composition. A dye, such as yellow dye, can also be added.

### Example 2. Composition of Nasal Spray Solution

Table 2 illustrates the components and concentrations that can be included and not limited to a nasal spray solution.

TABLE 2
Nasal Spray Composition

| Components | Contents in % of composition |
|---|---|
| Curcumin-L-Arginine co-amorphous | 0.001 to 0.10 |
| compound | |
| Surfactant 1 | 0.1 to 20 |
| Surfactant 2 | 0.1 to 20 |
| Mucoadhesive | 0.01 to 20 |
| pH Regulator | 0.1 to 5 |
| Isotonic solution | 0.8 to 1.1 |
| Vehicle | q.s. |
| Other additives q.s. | q.s. |
| Total Composition | 100% |

If required, an antibacterial agent, such as benzalkonium chloride, can be added in an amount of 0.1% of the composition. A dye, such as yellow dye, can also be added.

### Example 3. Nasal Spray Solution Formulations

Table 3 illustrates five formulations in nasal solution for atomization (spray) in accordance with the invention.

TABLE 3
Nasal solution formulations (gr per 100 ml)

| | Formulation 1 | Formulation 2 | Formulation 3 | Formulation 4 | Formulation 5 |
|---|---|---|---|---|---|
| Components | Content gr | Content gr | Content gr | Content gr | Content gr |
| Curcumin | 0.015 | 0.015 | 0.015 | 0.015 | 0.015 |
| L-Arginine | 0.015 | 0.015 | 0.015 | 0.015 | 0.015 |
| Glycerin | 10 | 10 | 10 | 10 | 10 |
| Propylene | 5 | 5 | — | — | — |
| glycol | | | | | |
| Polipolis | — | — | 5 | — | — |
| Polyethylene | 5 | — | 5 | — | — |
| glycol | | | | | |
| Carbopol | 0.3 | 5 | — | 0.3 | — |
| Sodium | 1 | — | — | — | — |
| alginate | | | | | |
| Hypromellose | — | — | — | 5 | 5 |
| Phosphate | q.s. | q.s. | q.s. | q.s. | q.s. |
| Regulator | | | | | |
| Vehicle | 0.9 | 0.9 | 0.9 | 0.9 | 0.9 |
| Isotonic | | | | | |
| solution | | | | | |
| Other | q.s. | q.s. | q.s. | q.s. | q.s. |
| additives q.s. | | | | | |

### Example 4. Nasal Spray Solution Formulations

Table 4 illustrates four formulations of nasal spray solution in accordance with the invention.

TABLE 4
Nasal solution formulations (gr per 100 ml)

| | Formulation 1 | Formulation 2 | Formulation 3 | Formulation 4 |
|---|---|---|---|---|
| Components | Content gr | Content gr | Content gr | Content gr |
| Curcumin-L- | 0.03 | 0.03 | 0.03 | 0.03 |
| Arginine co- | | | | |
| amorphous | | | | |
| compound | | | | |
| Glycerin | 10 | — | 10 | — |
| Propylene glycol | 5 | 5 | — | 5 |
| Polyethylene glycol | — | 5 | — | — |
| pH Regulator | q.s. | q.s. | q.s. | q.s. |
| Phosphates | | | | |
| Vehicle Isotonic | 0.9 | 0.9 | 0.9 | 0.9 |
| solution | | | | |
| (NaCl) | | | | |
| Other additives q.s. | q.s. | q.s. | q.s. | q.s. |

The illustrated formulations can be manufactured according to the following procedure:

The components of the solution are weighed.

In a mixing tank, the isotonic solution of sodium chloride is prepared.

In a separate container, the co-amorphous compound of curcumin and arginine is dissolved and the resulting solution is added to the mixing tank.

In a separate container, the selected surfactant(s) is/are prepared and then added to the mixing tank.

In a separate container, the mucoadhesive agent(s) are dissolved.

The mucoadhesive solution is added to the mixing tank.

In the mixing tank solution, the pH is adjusted between 6.5±0.5 with phosphate buffer solution.

The resulting solution is filtered through a 22 μm pore size membrane.

### Example 3. Stability Studies

Stability tests of the formulations of Table 4 were carried out under simulated extreme storage conditions: 40° C. and 75% relative humidity (RH) over a period of 8 weeks, evaluating the stability weekly.

The solutions showed no visible signs of instability in the times analyzed, so it can be concluded that it is stable.

### Example 4. Cytotoxicity Assays on Vero CCL81 Cells

A cytotoxicity assay was performed on a cell line isolated from African green monkey kidney epithelium (Vero CCL81 cells) by exposing the cells to different concentrations of the combination of curcumin and arginine.

Objective: To determine the cytotoxic activity of the product on Vero CCL81 cells by exposing the cells and observing the post-incubation monolayer.

Material and methods: Source: cell line isolated from African green monkey kidney epithelium. ATCC: Vero CCL81

Culture and maintenance: DMEM medium is grown with 10% fetal bovine serum (maintenance medium) and inoculation with DMEM high glucose with trypsin (10 ug/ml) is tested. Cell separation is done with trypsin.

Test conditions: Concentration of the test substance in the cytotoxicity test: 30, 10, 3, 1, 0.3, 0.1, 0.03, 0.01, 0.003 and 0.001 mg/mL. Contact time: 1 hour and 72 hours in cell culture.

Results: In the conformation of the cell monolayer and the state of the cells, it was observed that there were no alterations and changes in morphology. The combination of curcumin and arginine in the present invention has no cytotoxic effects since the cell monolayer can be seen without alterations in the morphology of the cells.

### Example 5. Antiviral Assay of Co-Amorphous Compound of Curcumin-Arginine Vs SARS-CoV2 in Vero CCL81 Cells

Objective: To determine the antiviral activity of curcumin-arginine co-amorphous against SARS-Cov2 in Vero CCL81 cells by means of a plaque assay.

### Material and Methods.

Source: Cell line isolated from African green monkey kidney epithelium. ATCC: Vero CCL81

Passage: Passage number 15-30

Culture and maintenance: DMEM medium is grown with 10% fetal bovine serum (maintenance medium) and inoculation with DMEM high glucose with trypsin (10 ug/ml) is tested. Cell separation is done with trypsin.

Test conditions: Curcumin-arginine concentration in the antiviral activity assay 30, 10, 3, 1, 0.3, 0.1, 0.03, 0.01, 0.003 and 0.001 mg/mL.

Contact time: 24 hours in cell culture.

Test replicates: 7 replicates per concentration.

Controls: Cellular control (DMEM high glucose medium with trypsin (10 ug/ml)), matrix control (1:2 dilution of DMSO with DMEM medium high glucose with trypsin (10 ug/ml)) and infection control (cells infected with 0.01 MOI SARS-Cov2 per 1 h). Witness replicas: 4 replicas for each witness.

### Procedure:

1. A stock of 100 mg/mL is prepared in 500 ul of DMSO, from this stock the necessary dilutions were made in the infection medium (DMEM medium high glucose+10 μg/ml of trypsin).

2. Vero CCL81 cell plates with 10,000 cells per well are prepared in 100 μL of maintenance medium and left to incubate for 24 hours at 37° C. with 5% CO2. The next day, a minimum cell confluence of 75% is confirmed.

3. Pre-infection treatment: The maintenance medium is removed from the Vero CCL81 cell monolayer and 100 μL of the different dilutions of the curcumin-arginine combination are added to each well for 24 h, then the curcumin-containing medium is removed and the viruses (MOI of 0.01) are added for one hour at 37° C. with 5% CO2. At the end of the incubation period, the monolayer is washed and maintenance medium is added and the plates are incubated for 48 hours at 37° C. with 5% CO2.

Co-treatment: Different concentrations of curcumin-arginine co-amorphous compound are mixed with SARS-CoV2 (MOI 0.01) and incubated for one hour at 37° C. Subsequently, the mixture of co-amorphous and virus is added to the monolayer of cells for one hour at 37° C. with 5% CO₂. At the end of the incubation period, the monolayer is washed and maintenance medium is added and the plates are incubated for 48 hours at 37° C. with 5% CO2.

Post-infection treatment: The maintenance medium is removed from the Vero CCL81 cell monolayer and the viruses are added to an MOI of 0.01 for one hour at 37° C. with 5% CO2. At the end of the infection period, the monolayer is washed and the different concentrations of the co-amorphous compound are added, the plates are incubated for 48 hours at 37° C. with 5% CO2.

Controls: Untreated Vero CCL81 cells will be the negative control, and Vero CCL81 cells infected with 0.01 SARS-CoV2 MOI in the absence of co-amorphous compound of curcumin-arginine, function as a positive control.

4. Once the treatments (pre-treatment, co-treatment, post-treatment and controls) are washed twice with PBS, fixed with 100 μL of 100% methanol and stained with a 1% crystal violet solution. The viral plaques formed are counted.

5. Viral activity is determined as the difference between viral plaques formed after curcumin treatment and untreated infection control.

### Results:

Curcumin-arginine co-amorphous compound exhibited antiviral activity before, during, and after SARS-Cov2 infection in the Vero CCL81 cell line.

The concentration of curcumin-arginine co-amorphous compound required to inhibit 100% SARS-CoV2 in the different treatment regimens (pre-treatment, co-treatment and post-treatment) was lower than the cytotoxic concentration in the Vero CCL81 cell line.

The concentration of curcumin-arginine co-amorphous compound required to inhibit 50% of the viral activity (EC50) of SARS-Cov2 was lower than the cytotoxic concentrations in the different treatment regimens (pre-treatment, co-treatment and post-treatment).

The combination of curcumin and arginine of the present invention was found to be surprisingly capable of reducing the cytopathic effects induced by SARS-CoV-2 by 100%.

### Example 6. Mucosal Irritability Test by the HET CAM Model

A mucosal irritability test was performed on the chorioallantoic membrane of the chicken embryo using the HET CAM (Hen's Egg Test-Chorioallantoic Membrane) model. The trial was performed with a composition of the two active agents curcumin+arginine in a 1:1 weight ratio testing the following concentrations: 30, 10, 3, 1, 0.3, 0.1, 0.03, 0.01, 0.003 and 0.001 mg/mL.

Procedure: The test compound was administered to the chorioallantoid membrane of the chicken embryo for a certain amount of time to determine if there is hemorrhage, lysis, and/or coagulation. The time of appearance was recorded in seconds during a maximum observation time. Controls such as NaOH will be administered. The irritation index is calculated.

It was determined that, according to the irritability classification for the HET-CAM method, the composition in the concentrations evaluated is in the Non-Irritant category, that is, it has a value less than 0.9 on the irritability scale of the HET-CAM test.

### Example 7. Antiviral Assay in a Syrian Hamster Model

Test conditions: concentrations of the test substance in the Syrian hamster antiviral assay: 30, 10, 3, 1, 0.3, 0.1, 0.03, 0.01, 0.003 and 0.001 mg/mL.

Procedure: The animals were randomized into the different test groups, acclimatized in an optimal time within the BSL-3 laboratory, anesthetized to be inoculated intranasally with the SARS-CoV-2 virus; in the same way, the combination of the two active agents arginine+curcumin was administered in a 1:1 weight ratio, at concentrations of 30, 10, 3, 1, 0.3, 0.1, 0.03, 0.01, 0.003 and 0.001 mg/mL. Monitoring of animals every day for 7 or more days. Measurement of SARS-CoV2 viral load by RT-PCR.

The main groups analyzed were:
Uninfected hamsters
Hamsters infected with SARS-CoV-2 without test compound
Hamsters infected with SARS-CoV-2 with test compound (inoculated at the same time, co-treatment)
Hamsters not infected with test compound and will subsequently be inoculated with SARS-CoV-2 (pre-infection treatment)
Hamsters infected with SARS-CoV-2 and subsequently inoculated with the test compound (post-infection treatment).

### Results:

The group of hamsters infected with SARS-CoV-2 with test compound has a significantly lower viral load compared to the viral load in the group of hamsters infected with SARS-CoV-2 without test compound, both in post-infection treatment and in pre-infection treatment.

### Example 8. Phase III Clinical Study

This is a randomized, double-blind, controlled clinical trial to evaluate the efficacy and safety of a nasal solution of the compound of curcumin and arginine, as an adjuvant for the treatment of mild and symptomatic COVID-19 infection.

Primary Objective: To evaluate the efficacy of curcumin-arginine nasal solution on SARS-CoV-2 RNA viral load compared to a control group on days 2, 4, 6, 10, and 14 of the intervention in patients with mild COVID-19.

Secondary objectives: To evaluate the effect of nasal solution as an adjuvant on symptoms of mild COVID-19. Assess the safety of the nasal solution.

Experimental design: Randomized, double-blind clinical trial to evaluate efficacy and safety.

Number of subjects: 80 subjects (40 in each treatment arm)

Diagnosis and main inclusion criteria: Men and women with a complete SARS-CoV-2 vaccination schedule, adults between 18-80 years of age, patients with mild COVID-19 confirmed by a positive SARS-CoV-2 test by RT-PCR obtained by nasopharyngeal swab. Subjects with symptoms of mild COVID-19 disease, according to the FDA severity classification: fever (temperature >37.3° C.), cough, pharyngeal pain, general malaise, headache, myalgias, nausea, vomiting, diarrhea, loss of smell (anosmia), loss of taste (ageusia), SpO2≥93%, No shortness of breath or dyspnea.

Material: Treatment A: arginine curcumin, Nasal solution, Route of administration, nasal. Dosage: Up to 6 times a day (one or two applications to each nostril) for 14 days. Treatment B: Placebo, Nasal solution, Route of administration, nasal. Dosage: 6 times daily (two applications in each nostril) for 14 days

All patients will be standardized for symptomatic treatment.

Duration of treatment: 14 days

Efficacy variables: Primary: Decreased relative expression of SARS-CoV-2 RNA load. Secondary: 1) Time to resolution of clinical symptoms per treatment; 2) Proportion of patients with clinical cure (no clinical symptoms) at 2, 4, 6, 10 and 14 days.

Methodology. Treatment with curcumin-arginine nasal solution or placebo begins on day 1, and assignment to treatment will be randomized. Participants are sampled at times 2, 4, 6, 10 and 14 in the mornings prior to the application of the treatment, to perform the RT-PCR-Quantitative test. Symptoms are monitored from a patient's diary and through video calls by the researcher on days 2, 6 and 10 of treatment, as well as identification of adverse events, tolerance, adherence, lack of efficacy and worsening of health status. The study concludes with a face-to-face visit on the 14th to discharge the patient and carry out the closing visit.

Statistical methods: Continuous variables are expressed as means and standard deviation and dichotomous variables are expressed as frequency and percentage. The distribution of variables is evaluated using statistical tests.

### Results:

In the present study, the composition of curcumin-arginine in nasal solution was shown to have a strong antiviral effect against SARS-CoV-2. Patients who received the nasal solution, starting on day 2, showed a decrease in RNA load. These findings highlight curcumin as an antiviral compound against SARS-CoV-2. The nasal solution also showed in cell cultures that the combination of curcumin and arginine is not cytotoxic and does not modify cell morphology.

Other studies have shown that high amounts of curcumin are safe. The present invention curcumin L-arginine employs amounts drastically below those amounts, so the antiviral, anti-infective effect shown by the invention is surprising.

### NOVELTY OF THE INVENTION

Having described the present invention as above, it is considered as novelty and, therefore, the contents of the following inventions are claimed as property.

1-29. (canceled)

30. A pharmaceutical composition for nasal delivery, characterized by comprising a solution of curcumin and arginine formed from a co-amorphous compound of curcumin and arginine having a curcumin:arginine molar ratio from 1:2 to 2:1, and pharmaceutically acceptable excipients, wherein the solution has a pH of 5 to 8 and wherein the pharmaceutically acceptable excipients include: (a) at least one buffering agent selected from phosphate salts, carbonate salts and combinations thereof; (b) at least one surfactant selected from propylene glycol, polyethylene glycol, PEG400, PEG3500, polyoxyl stearate 400, polysorbate, glycerin, diethylene glycol monoethyl ether (transcutol), polyoxyethylene-polyoxypropylene (lutrol), cetylpyridinium chloride (CPC), cetylpyridinium bromide (CPB), Tween 80; Poloxamer 407 and combinations thereof; (c) at least one mucoadhesive agent selected from methyl vinyl ether copolymer, hydroxypropyl methylcellulose, sodium carboxymethylcellulose, carbopol, sodium alginate, potassium alginate, magnesium alginate, modified starches, polyvinylpyrrolidone, beta-glucans, chitosan, Eudragit and combinations thereof; and (d) vehicles selected from isotonic saline solution, water and seawater.

31. The pharmaceutical composition in accordance with claim 30, wherein the co-amorphous compound has a curcumin to arginine molar ratio selected from 1:2, 2:1, and 1:1,

32. The pharmaceutical composition in accordance with claim 30, wherein the co-amorphous compound has a curcumin to arginine molar ratio of 1:2.

33. The pharmaceutical composition in accordance with claim 30, wherein the solution has a pH between 6 and 8.

34. The pharmaceutical composition in accordance with claim 30, wherein the pharmaceutically acceptable excipients also include solubilizers, emulsifiers, binders, preservatives, lubricants, viscosifiers, colorants, stabilizers and/or adjuvants.

35. The pharmaceutical composition in accordance with claim 34, wherein the composition additionally includes benzalkonium chloride.

36. The pharmaceutical composition in accordance with claim 30, wherein the buffers are selected from KH2PO4, K2HPO4 and Na2HPO4.

37. The use of the pharmaceutical composition in accordance with claim 30, in the manufacture of a medicament useful in the treatment of an infectious disease, where the infectious disease is an infectious respiratory disease or an airborne systemic infectious disease caused by an etiologic agent.

38. The use of the pharmaceutical composition in accordance with claim 37, wherein the etiologic agent of the disease is a bacterium selected from the group consisting of group A streptococci, Mycoplasma pneumoniae, Mycoplasma hominis, Streptococcus pneumoniae, Corynebacterium diphtheriae, Streptococcus pyogenes, and Haemophilus influenzae.

39. The use of the pharmaceutical composition in accordance with claim 37, wherein the etiologic agent of the disease is a virus selected from the group consisting of rhinovirus, coronavirus, parainfluenza virus, Epstein-Barr virus, cytomegalovirus, herpes simplex virus, adenovirus, influenza virus, respiratory syncytial virus, MERS, enterovirus, SARS-CoV-1 and SARS-CoV-2.

40. The use of the pharmaceutical composition in accordance with claim 39, wherein the virus is SARS-CoV-2.

41. The pharmaceutical composition in accordance with claim 30, wherein the composition is adapted to be administered in each nostril at least twice a day.

42. The pharmaceutical composition in accordance with claim 30, wherein the composition is adapted to be administered into each nostril at least three times a day.

43. The pharmaceutical composition in accordance with claim 30, wherein the composition is adapted to be administered into each nostril two to six times a day.

44. The pharmaceutical composition in accordance with claim 30, wherein the composition is adapted to be administered into each nostril in an amount of 90 to 180 μL.

45. The use of a curcumin-arginine co-amorphous compound having a molar ratio of curcumin to arginine 1:2, in the manufacture of a medicament useful in the treatment of an infectious disease caused by SARS-CoV2 virus, respiratory syncytial virus, adenovirus or influenza virus, wherein the medicament is in the form of a solution and is adapted for nasal administration.

46. A curcumin-arginine co-amorphous compound having a molar ratio of curcumin to arginine of 1:2, for use in the treatment of an infectious disease caused by SARS-CoV2 virus, influenza virus, adenovirus and respiratory syncytial virus, wherein co-amorphous compound is formulated in a solution and is adapted for nasal administration.

47. The co-amorphous compound in accordance with claim 46, wherein the co-amorphous compound is adapted to be administered into each nostril at least twice a day.

48. The co-amorphous compound in accordance with claim 46, wherein the co-amorphous compound is adapted to be administered into each nostril at least three times a day.

49. The co-amorphous compound in accordance with claim 46, wherein the co-amorphous compound is adapted to be administered into each nostril from two to six times a day.

50. The co-amorphous compound in accordance with claim 46, wherein the co-amorphous compound is adapted to be administered into each nostril in an amount of 90 to 180 UL.

51. The use of co-amorphous compound in accordance with claim 45, wherein the medicament is formulated to be administered into each nostril at least twice a day.

52. The use of co-amorphous compound in accordance with claim 45, wherein the medicament is formulated to be administered into each nostril at least three times a day.

53. The use of co-amorphous compound in accordance with claim 45, wherein the medicament is formulated to be administered into each nostril from two to six times a day.

54. The use of co-amorphous compound in accordance with claim 45, wherein the medicament is formulated to be administered into each nostril in an amount of 90 to 180 μL.
